(19) United States
(12) Patent Application Publication
(10) Pub. No.: US 2015/0351968 A1
(43) Pub. Date: Dec. 10, 2015

(54) Ear Brush

(71) Applicant: Kimberly Shane, Quincy, MA (US)

(72) Inventor: Kimberly Shane, Quincy, MA (US)

(21) Appl. No.: 14/296,787

(22) Filed: Jun. 5, 2014

(51) Int. Cl. A61F 11/00

(52) U.S. Cl. CPC A61F 11/006

(57) ABSTRACT

An ear brush for removal of cerumen and other debris from the ear, and for relief of inner ear irritations. The ear brush is comprised of a rod, with a flexible end, characterized by attached perpendicular flexible bristles. The securely attached bristles are lined in parallel form, on one or both ends of the rod. The rod end is made of a durable and flexible material to gently follow the inner contours of the ear.

### BACKGROUND OF THE INVENTION

The present invention concerns an ear brush for the removal of earwax, also known as cerumen, from the ear. Earwax secretion is a natural function, created by the body as a protectant for the inner ear and eardrum. However, earwax buildup in the ear can be a pesky and challenging problem, leading to itchiness, blocked hearing, and frustration, especially considering the unique curves of different ears.

On the market, cotton swabs are the most commonly used method employed to attempt to remove earwax. Unfortunately, the rounded end shape of cotton swabs and the useless cotton tip rarely actually remove the earwax from the ear and can lead to further impaction of the wax deeper into the ear. Furthermore, the cotton fibers on the end of the swab may also get dislodged into the ear, creating an even more troublesome situation.

As the cotton swab is the most common method on the market presently used for earwax removal, several variations on the cotton swab have been proposed including those described in U.S. Pat. No. 2,510,961; U.S. Pat. No. 2,876,501; U.S. Pat. No. 5,147,288; E.P. 0120235 A1; and E.P. 2505172 A1, which have been designed in effort to better remove earwax, but all still fail to prevent the impaction of the wax against the surface of the inner ear.

Other previously invented earwax removal systems include those described in U.S. Pat. No. D490523, a hooped rod for ear cleaning; Pub. No. 2006/0085018 A1, a coiled wand for ear cleaning; and U.S. Pat. No. 6155987, a funnel-shaped speculum to aid in the removal of earwax. Several advantages of the present invention over these include less potential for ear injury, less chance of earwax impaction from use, greater ability for wax removal, and all in an inexpensive, convenient, and safe product.

### SUMMARY

The primary purpose of the present invention is to provide safe and easy removal of earwax from an ear and be a solution to inner ear itches, blockage, and irritation. Aspects of the invention include a stiff but flexible handle rod, with a flexible end of bristles on at least one end of said rod, that curve with the ear. As mentioned, advantages of different aspects of the invention include a better shape to fit the contours of an ear; a more effective method of wax removal; and a decreased chance for wax impaction against the walls of the ear.

### BRIEF DESCRIPTION OF THE DRAWINGS

FIG. 1 shows the front, vertical view of the ear brush, bristles and rod.

FIG. 2 shows the end-of-the rod fixated flexible bristles.

FIG. 3 illustrates the bendable nature of the rod end, flexing with the curvature of the ear.

### DESCRIPTION OF THE INVENTION

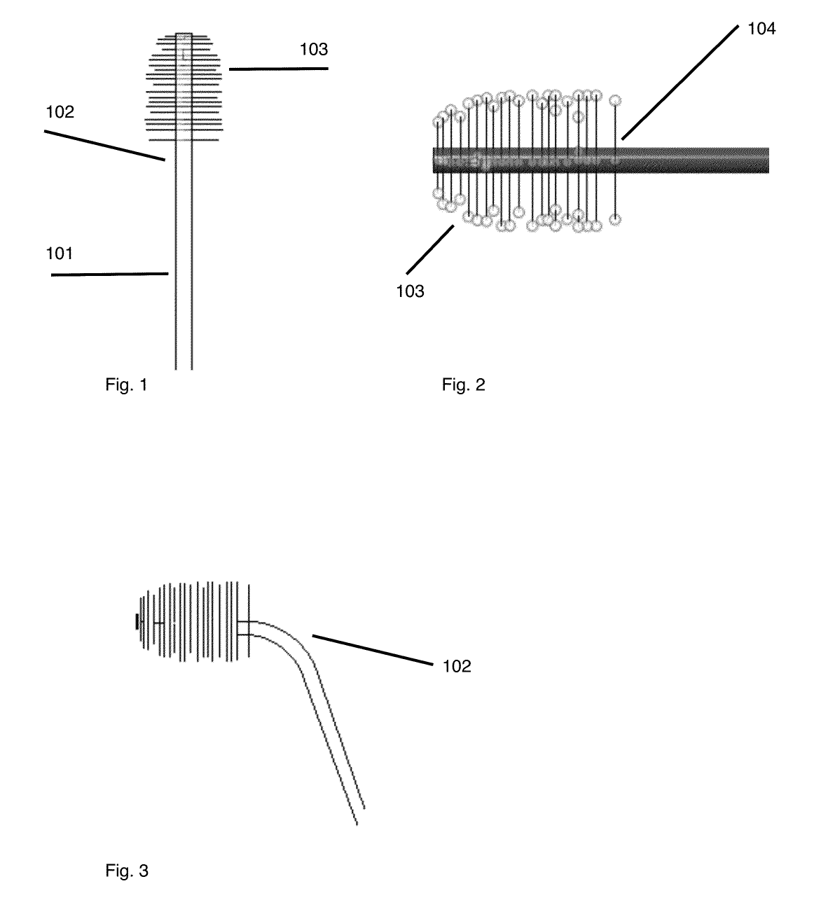

The present invention provides an ear brush for removing debris from the ear and relieving inner ear itchiness. FIG. 1 illustrates the vertical side view of the ear brush, wherein the length of the brush rod is variable, it has extending, flexible bristles attached the rod end, with the end being flexible in nature to move with the contours of an ear. The rod 101 can consist of any number of materials that are sturdy and allow for the grip of a hand, such as plastic, paper, cardboard, rubber, etc., and can vary in length, likely between 6 cm and 9 cm in length.

Protruding from the end of the rod 102 are a multitude of bristles or fibers 103 that are likely around 0.5 cm or so in length. These bristles are made of a soft material, such as nylon or polyester, so not to harm the inner ear, but firm enough so as to collect cerumen, dry flakes, and/or buildup, and relieve, usually related, inner ear itchiness. The end rod 102 is made of a flexible but durable material, such as silicone or rubber, to allow gentle movement of the rod end and bristles as it is inserted into the ear and pressed against the inner ear tissue or buildup.

The bristles 103 are connected to the rod end 102 at individual connection points 104 that prevent any unintended dislodging of the bristles from the rod into the ear. The bristles are firmly attached to the rod end 102 to be able to endure the ear brush's use in an ear full of debris and heavy motion.

From the description above, a number of advantages of the ear brush become evident:
(a) Increased ability to remove cerumen, dryness, or other debris from the ear.
(b) Increased ability to relieve ear itches or irritation.
(c) Decreased or eliminated likelihood of lost fibers lodged into the inner ear as seen with the current inner ear cerumen removal swabs.
(d) Increased ability for flexible insertion into the ear to follow the unique contours of an ear.
(e) Decreased chance of cerumen or debris impaction into the inner ear with the unique flexible rod end with attached fibers being more ear compatible than the current inner ear cerumen removal swabs with rounded tips that cause impaction. (f) Decreased likelihood of ear injury by use for cerumen removal because of the unique flexible rob that moves with the contours of the ear and the soft attached bristles that graze the delicate ear tissue.

What is claimed is:

1. An ear brush for the removal of earwax and relief of inner ear irritations, which comprises of a rod having rod ends on at least one of its ends, characterized in that a plurality of soft bristles are attached firmly to all sides of a rod end.

2. An ear brush as described in claim 1, characterized in that the bristles on the rod end are generally perpendicular to the rod end and parallel to the other bristles.

3. An ear brush according to any of the claims 1 to 2, characterized in that the rod end if made of flexible material.
